(19) United States
(12) Patent Application Publication
(10) Pub. No.: US 2019/0262476 A1
(43) Pub. Date: Aug. 29, 2019

(54) METHOD FOR MEASUREMENT AND CONTROL OF INTRACULAR VEGF CONCENTRATION

(71) Applicant: JUSTUS-LIEBIG-UNIVERSITAET GIESSEN, Giessen (DE)

(72) Inventors: Birgit Lorenz, Laubach (DE); Tobias Wimmer, Giessen (DE); Knut Stieger, Lich (DE)

(21) Appl. No.: 16/079,431

(22) PCT Filed: Feb. 20, 2017
(86) PCT No.: PCT/EP2017/053803

§ 371 Date: Aug. 23, 2018

(30) Foreign Application Priority Data
Feb. 23, 2016 (EP) 16156886.0

(51) Int. Cl. A61K 49/00; G01N 33/68

(52) U.S. Cl. CPC A61K 49/0047; G01N 2800/16; G01N 2333/475; G01N 33/6893

(57) ABSTRACT

The invention describes a new method for in vivo measurement and control of intraocular VEGF concentration using bioluminescence resonance energy transfer (BRET) of a VEGF-binding biosensor. Furthermore, the method is suitable for highly sensitive in vitro determination of VEGF concentration from a small sample volume.

### BACKGROUND OF THE INVENTION

Most retinal neovascular disorders are caused by upregulation of vascular endothelial growth factor (VEGF) expression, which leads to an uncontrolled formation of new, immature blood vessels in the eye. VEGF is a homodimeric heparin-binding glycoprotein with pro-angiogenic properties, stimulating migration and proliferation of micro- and macrovascular endothelial cells. The VEGF family consists of at least six subgroups named VEGF-A to VEGF-E and the placental growth factor. VEGF-A is the most prominent and potent isoform and key regulator in physiological angiogenesis as well as abnormal vascularization (neovascularization [NV]).

The expression of VEGF is upregulated under hypoxic conditions and by various cytokines. Ocular diseases with NV as a typical pathological feature include the neovascular form of age-related macular degeneration (wet/exudative AMD), diabetic macular edema (DME) in patients with diabetic retinopathy, retinal vein occlusion (RVO), and retinopathy of prematurity (ROP). The neovascularization is mostly caused through the effects of VEGF, inducing a phenotypic switch of endothelial cells. VEGF-A stimulates angiogenesis and NV through the binding to VEGF receptors (VEGF-R) located on endothelial cell surfaces, thus activating intracellular signaling pathways.

An effective way to inhibit VEGF-mediated activation of VEGF-Rs is to neutralize VEGF molecules before binding to the receptor through interaction with a VEGF-binding protein (anti-VEGF). With the advent of production techniques like the phage display technology and recombinant DNA technology, the generation of anti-VEGF molecules became possible. These molecules include whole antibodies (bevacizumab, Avastin®), antigen-binding fragments [F(ab)s] (ranibizumab, Lucentis®), or soluble molecules containing parts of the receptor-binding domain of the VEGF-R (aflibercept, EYLEA®).

Ranibizumab, the humanized F(ab) of the original whole IgG antibody bevacizumab, is inactivating VEGF due to the binding to the receptor-binding sites of all VEGF-A isoforms. It was uniquely designed for the treatment of NV in the eye and is FDA and EMA approved for use in AMD, DME, and RVO.

The pivotal disadvantage of all anti-VEGF molecules is the short half-life in the human eye (e.g., t1/2=7.19 days for ranibizumab). This impedes repeated injections to provide sustained VEGF blockade. However, such repeated injections of anti-VEGF molecules are very expensive and may have severe side effects like intraocular inflammation or retinal detachment. As to the state of the art, therapeutic interventions involving administration of anti-VEGF molecules are not correlated so far with the actual prevalent VEGF concentration but are solely based on the diagnosis of a retinal neovascular disorder.

According to the state of the art, tetracycline-inducible (TetOn) vectors have been constructed that encode single chain variable fragments (scFv) of anti-VEGF molecules (anti-VEGF-scFv), like for example Ra02, which is based on Ranibizumab and expressed as one single molecule.

As known from the state of the art, it is possible to measure VEGF concentration ex vivo, either with samples from blood or from aqueous liquid that was received by dotting of the eye, using enzyme-linked immunosorbent assay (ELISA) formats. However, these methods have some major disadvantages: First, it is not clear whether the VEGF concentration in blood correlates with the VEGF concentration in the eye; second, dotting of the eye may cause severe afflictions like endophthalmitis that may lead to complete loss of sight; and third, all commercially available ELISA formats need high sample volumes (>5 μl) and have a detection limit in the range of picogram per milliliter (pg/ml). Plasma VEGF concentrations in healthy volunteers and patients with neovascular disorder and different forms of cancer are often below the current limit of detection (5-10 pg/ml). However, this factor is crucial in the pathogenesis of these disorders and precise knowledge about the concentrations in the circulation is crucial. A method for minimally invasive in vivo measurement of VEGF concentration in the eye of patients with retinal neovascular disorders is so far not known.

### Technical Problem

There is a need for a minimally invasive method for determination of intraocular VEGF concentration that allows for a decision if therapeutic intervention with anti-VEGF antibodies of patients with a retinal disorder, like age-related macular degeneration, diabetic macular edema, diabetic retinopathy, retinopathy of prematurity, or retinal vein occlusion, is necessary due to the measured prevalent VEGF concentration.

Moreover, there is a need for a method combining both, minimally invasive intraocular in vivo measurement of VEGF concentration and control of VEGF concentration by synthesis of anti-VEGF molecules in vivo that is dependent on the measured VEGF concentration.

Furthermore, the state of the art lacks a highly sensitive assay in the range of femtogram per milliliter (fg/ml) for in vitro determination of VEGF concentration with a small sample volume.

### Solution of the Problem

The aforementioned technical problem is solved by a method using a biosensor that provides an increased fluorescent BRET signal upon binding of VEGF. For measurement of VEGF concentration in vivo, the biosensor is encapsulated in an eye-implantable, permeable microcapsule, microparticle, microbead, or gel. Additionally, for control of VEGF concentration in vivo, a doxycycline-inducible vector for synthesis of anti-VEGF molecules that is transduced into eukaryotic cells is also encapsulated therein. Moreover, the invention provides a method for highly sensitive determination of VEGF concentration in the range of femtogram per milliliter in vitro with a small sample volume of 1 to 10 μl.

### DESCRIPTION OF THE INVENTION

In one aspect, the invention provides a method for measurement and control of intraocular VEGF concentration, which comprises the following steps:
addition of a Renilla luciferase substrate to VEGF-binding biosensor molecules each comprising an anti-VEGF single chain variable fragment (anti-VEGF-scFv) with Renilla luciferase fused to its N-terminus and a fluorescent protein or peptide fused to its C-terminus in a liquid containing VEGF,
measurement of bioluminescence resonance energy transfer (BRET) signal depending on binding of prevalent VEGF to the biosensor molecules as an indicator for VEGF concentration,
induction of expression of anti-VEGF molecules by addition of doxycycline to a vector encoding anti-VEGF molecules that is transduced into eukaryotic cells.

According to the present invention, in the first step of the method prevalent VEGF molecules bind to the VEGF-binding biosensor molecules, which are chimeric proteins that comprise each an anti-VEGF single chain variable fragment (anti-VEGF-scFv, VEGF binding domain) with Renilla luciferase fused to its N-terminus and a fluorescent protein or peptide fused to its C-terminus. Such biosensors are known from the state of the art.

In a preferred embodiment, the anti-VEGF-scFv is Ra02, which is derived from Ranibizumab, the Renilla luciferase is Renilla luciferase mutant 8 (RLuc8), and the fluorescent protein is GFP2, YFP, eYFP, TurboYFP, or a peptide or derivative or mutant thereof; all of these molecules are known to the skilled worker.

If VEGF is present, it binds to the anti-VEGF-scFv part of the chimeric protein. Subsequently, binding of VEGF triggers a conformational change (ligand-induced conformational rearrangement) of the chimeric protein. In the presence of Renilla luciferase substrate Coelenterazine, e.g. EnduRen™ or ViviRen™, this conformational change is generating an increase of a BRET signal, which is mediated through radiationless energy transfer, based on dipole-dipole interaction, from the N-terminally located Renilla luciferase as the signal donor to the C-terminally located fluorescent protein as the signal acceptor. The BRET signal is a quotient of the intensity of the emitted radiation of the fluorescent protein and the intensity of the emitted radiation of the Renilla luciferase that is due to substrate conversion (FIG. 1). The intensity of the BRET signal directly correlates with the VEGF concentration: a higher VEGF concentration leads to a higher BRET signal. The BRET technology is described in the state of the art.

For performance of the method in vivo, i.e. in the eye of a patient with a retinal neovascular disorder, the VEGF-binding biosensor as described herein according to the present invention is encapsulated in an insert like a microcapsule, microparticle, microbead, or gel, that is permeable for VEGF, Renilla luciferase substrate, doxycycline, and anti-VEGF molecules, but is not permeable for VEGF-anti-VEGF antigen-antibody complexes or VEGF that is bound to the VEGF-binding biosensor.

In a preferred embodiment, the insert is made from alginate. This insert can be implanted into the eye of the patient, which enables a permanent, minimally invasive measurement of VEGF concentration directly in the eye via BRET signal using a device that is able to detect the BRET signal through the vitreous body and the front part of the eye (FIG. 2). Such devices are commercially available which use an appropriate software, like ImageJ, for instance, to analyze the image data. For in vivo measurement, the Renilla luciferase substrate Coelenterazine is administered intravenously or orally to the patient. Based on the measured VEGF concentration, a decision can be made if a therapeutic intervention by administration of anti-VEGF molecules is necessary or not (Example 1).

In order to enable a minimally invasive therapy avoiding dotting of the eye, a vector encoding anti-VEGF molecules that is transduced into a eukaryotic cell line is additionally encapsulated in the insert (FIG. 2, SEQ ID No. 2). In a preferred embodiment, the vector encoding anti-VEGF molecules is TetOn-Ra02 (SEQ ID No. 2) as known from the state of the art. Expression of anti-VEGF molecules like Ra02 from this vector is induced by addition of a tetracycline, preferably doxycycline. In a preferred embodiment, doxycycline is administered orally to the patient at doses as described in the state of the art (Example 2).

For performance of the method in vitro, a vector encoding a VEGF-binding biosensor according to the invention is transfected into eukaryotic cells. In a preferred embodiment, the eukaryotic cells are HEK-293 cells and the VEGF-binding biosensor is RLuc8-Ra02-GFP2 (SEQ ID No. 1). In the cells, the biosensor is expressed within 48 hours and can be subsequently isolated from the cells, as described in example 3. Afterwards, an aliquot of the biosensor sample is incubated with samples from which the VEGF concentration has to be determined. These samples are from blood or from aqueous liquid of the eye of a patient whose VEGF concentration in the eye should be determined. In parallel, the biosensor is incubated with appropriate positive controls (VEGF serial dilution) or negative controls (PBS, RLuc8-Ra02). After addition of Renilla luciferase substrate Coelenterazine, the BRET ratio is measured with a plate reader in dual luminescence mode using the filter sets magenta and green, the ratio is normalized with the negative samples, and the VEGF concentration of the samples is determined by linear regression (Example 3, FIG. 1).

Surprisingly it is found in the present invention, that much lower VEGF concentrations can be measured by use of the VEGF-binding biosensor as described herein as with assays known from the state of the art. The new method is suitable for measurement of VEGF concentrations from 100 fg/ml or up to 10 ng/ml (FIG. 3), whereas commercially available assays have a detection limit of at least 1.7 pg/ml.

### EXAMPLES

The examples below illustrate the present invention.

### Example 1: Determination of Intraocular VEGF Concentration In Vivo

According to the present invention, the intraocular VEGF concentration in vivo is determined via the following steps. First, the substrate for Renilla luciferase, Coelenterazine, is administered to the patient whose VEGF concentration should be measured in the eye and to whom the VEGF-binding biosensor, for example RLuc8-Ra02-GFP2 (SEQ ID No.1), or RLuc8-Ra02-YFP, or RLuc8-Ra02-eYFP, or RLuc8-Ra02-TurboYFP, encapsulated in an insert, e.g. a microbead made from alginate, according to the present invention (FIG. 1, FIG. 2) has been implanted previously into the eye. Coelenterazine is commercially available, e.g. as EnduRen™ or ViviRen™ Live Cell Substrate (Promega), and is administered intravenously or orally, respectively, at doses that are known to the skilled worker. Subsequently, at an appropriate time after administration, the BRET ratio is measured using filters absorbing light at wavelengths of below 450 nm (signal from Renilla luciferase) and 500-550 nm (BRET signal from fluorescent protein or peptide), respectively, in a device for detection of luminescence using an appropriate image analysis software, like e.g. ImageJ. Such devices and softwares are known to the skilled worker. The measured delta BRET ratio correlates with the prevalent VEGF concentration in the eye of the patient and is determined from the BRET ratio by linear regression.

### Example 2: Induction of Anti-VEGF Synthesis In Vivo

According to the invention described herein, the insert harbouring the VEGF-binding biosensor may additionally encapsulate a tetracycline-inducible (TetOn) vector encoding anti-VEGF molecules that is transduced into HEK-293 cells (FIG. 2). This vector is TetOn-Ra02 that has been previously described (Wimmer et al., Functional Characterization of AAV-Expressed Recombinant Anti-VEGF Single-Chain Variable Fragments In Vitro. J Ocul Pharmacol Ther. 2015 June; 31(5):269-76) (SEQ ID No. 2). Expression of anti-VEGF molecules from TetOn-Ra02 is induced by oral administration of doxycycline to the patient with doses according to the state of the art (0.5 to 10 mg/kg body weight). The newly synthesized anti-VEGF molecules bind to VEGF that is present in the eye and therefore reduces the concentration of free VEGF. The reduction of VEGF concentration can be determined at an appropriate time after administration of doxycycline by the method described in example 1 according to the present invention.

### Example 3: Determination of Intraocular VEGF Concentration In Vitro

According to the invention presented herein, for determination of intraocular VEGF concentration in vitro, in a first step for example 6 μg of a vector encoding VEGF-binding biosensor molecules, for example RLuc8-Ra02-GFP2 (SEQ ID No. 1), are transfected in a 6-well with Lipofectamine 3000 (Invitrogen) into eukaryotic HEK-293 cells. This method is known to the skilled worker. RLuc8-Ra02-GFP2 comprises an anti-VEGF single chain variable fragment (anti-VEGF-scFv; Ra02) with Renilla luciferase (RLuc8) fused to its N-terminus and a fluorescent protein (GFP2) fused to its C-terminus. Afterwards, expression of RLuc8-Ra02-GFP2 biosensor molecules is allowed for 48 hours in an incubation chamber at 37° C. and 5% CO₂. Expression is then verified by luciferase activity assay and fluorescence microscopy; both techniques are known from the state of the art. Subsequently, the newly synthesized biosensor molecules RLuc8-Ra02-GFP2 are isolated from the HEK-293 cells by use of 150 μl per well Renilla Luciferase Assay Lysis Buffer (Promega) followed by two steps of freezing in liquid nitrogen and thawing. Afterwards the samples are centrifuged for 5 min at 14,000×g and 4° C. in order to get rid of any left cell particles. The soluble fraction then harbors the biosensor molecules. In parallel, serial dilutions ranging from 10 ng/ml down to 1 fg/ml of VEGF are made in phosphate-buffered saline (PBS) as positive control. Furthermore, serial dilutions in PBS are made with 1 or up to 10 μl of samples from which the VEGF concentration shall be determined. For determination of the VEGF concentration, 10 μl of the biosensor fraction is incubated at 4° C. overnight with 10 μl each of either the serial VEGF dilutions as positive controls, or the diluted samples from which the VEGF concentration should be determined, or PBS as a negative control, or a RLuc8-Ra02 construct lacking GFP2 at the C-terminus as negative control. Afterwards, 100 μl of the Renilla luciferase substrate Coelenterazine 400a (Nanolight, Inc.; 63 μM) is added to each of the samples to start the BRET assay. The BRET ratio is measured with a plate reader (Tecan Infinite 1000Pro) in dual luminescence mode using the filter sets magenta and green. The BRET ratio is normalized using the negative sample (delta BRET ratio). The correlating VEGF concentration, which is determined by linear regression, is displayed on the x-axis, and the delta BRET ratio is shown on the y-axis.

As can be seen in FIG. 3, dependent on the VEGF concentration, the BRET ratio is 0.785±0.039. The VEGF binding capacity per 1×10⁶ RLU (relative light units) is 20.29 pg±6.60 pg. The linear range of the VEGF dependent BRET ratio change is 100 fg/ml up to 10 ng/ml for the RLuc8-Ra02-GFP2 biosensor.

### DESCRIPTION OF THE DRAWINGS

FIG. 1. Antigen-induced conformational change of the VEGF-binding biosensor according to the present invention and BRET assay. If VEGF molecules are present, VEGF binds to the Ra02 parts (Ra02-L and Ra02-H) of the biosensor molecule and therefore induces a conformational change of the biosensor molecule. Thus, in the presence of Renilla luciferase substrate Coelenterazine, the Renilla luciferase RLuc8 at the N-terminus of the biosensor molecule emits radiation as a donor (BRET signal 1) that is then accepted by a fluorescent protein or peptide, e.g. GFP2, eGFP, eYFP, or TurboYFP, at the C-terminus of the biosensor molecule. The acceptor molecule then emits itself radiation at another wavelength than the donor (BRET signal 2). Both BRET signals are measured using a device using appropriate filters (e.g. magenta for BRET signal 1 and green for BRET signal 2) and a software that determines the BRET ratio. Then the BRET ratio is used for determination of the VEGF concentration by linear regression methods. The different parts of biosensor molecule (RLuc8, Ra02, and the fluorescent protein or peptide) are either fused directly to each other or are separated by proline (Pro) or 4× glycine (4Gly) linkers.

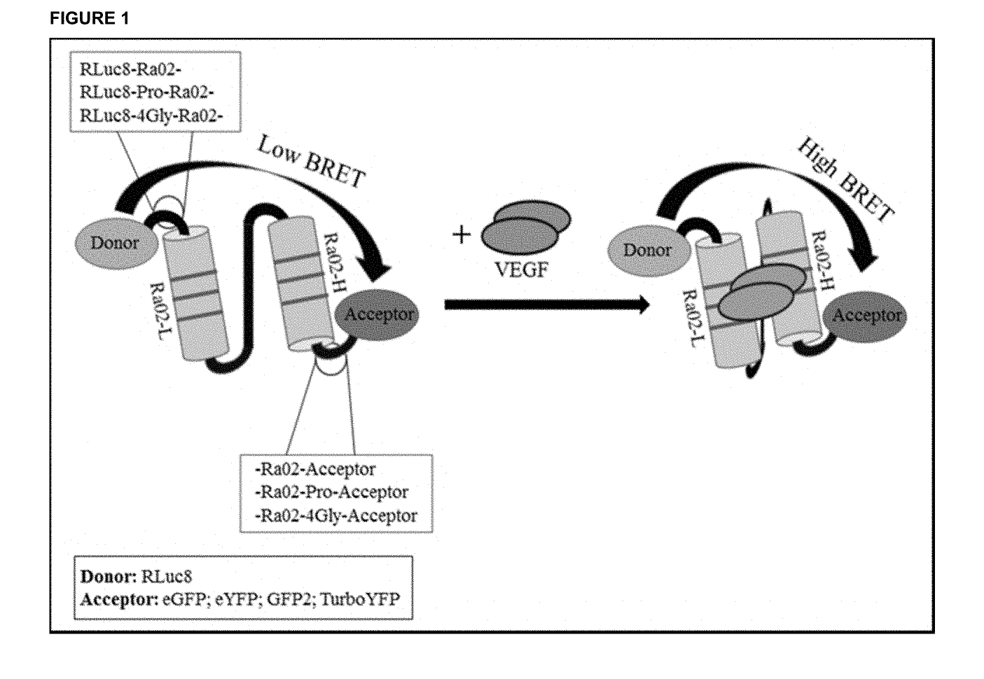

FIG. 2. Insert for implantation into the eye of a patient whose VEGF concentration in the eye should be measured in vivo. The insert is a microcapsule, microparticle, microbead, or gel, for example made from alginate, that is permeable for VEGF, Renilla luciferase substrate Coelenterazine, doxycycline, and anti-VEGF molecules, but is not permeable for VEGF-anti-VEGF antigen-antibody complexes and VEGF bound to the VEGF-binding biosensor. The insert encapsulates VEGF-binding biosensor molecules, like e.g. RLuc8-Ra02-GFP2 (SEQ ID No. 1), and additionally a doxycycline-inducible vector, TetOn-Ra02, that encodes anti-VEGF molecules and which is transduced into eukaryotic cells. A In the absence of free VEGF molecules, RLuc8 may convert its substrate Colenterazine and emit radiation, but this radiation can be transferred via BRET to the acceptor GFP only at a very low level. B In the presence of free VEGF molecules, the biosensor molecules undergoes a conformational change upon binding of VEGF to Ra02 of the biosensor. As a consequence, in the presence of Coelenterazine, the radiation of RLuc8 is transferred to the acceptor GFP, which then itself emits radiation. Both signals can be measured with an appropriate device and the BRET ratio as well as the VEGF concentration can be determined as described herein. If the VEGF concentration is too high, synthesis of anti-VEGF molecules can be triggered by administration of doxycycline to the patient. Doxycycline then induces expression of anti-VEGF molecules from the vector TetOn-Ra02 in the eukaryotic cells that are also encapsulated in the insert. Newly synthesized anti-VEGF molecules are small enough to leave the insert and to bind to free VEGF that is present in the eye of the patient.

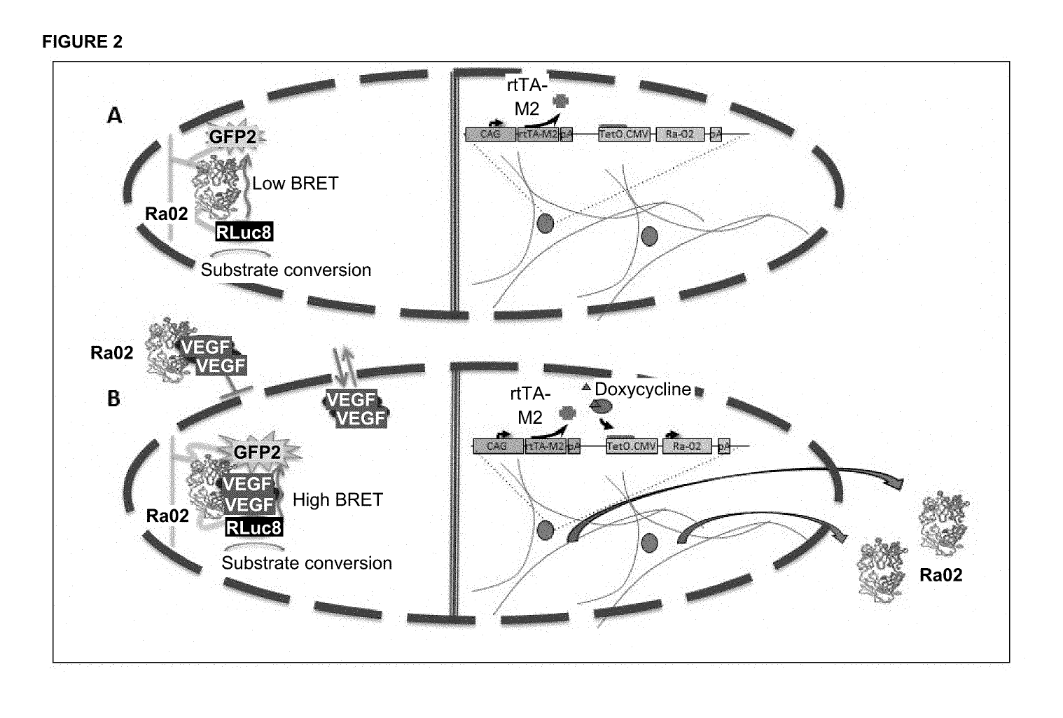

FIG. 3. Change of BRET ratio in dependence of the VEGF concentration. The VEGF concentration is displayed on the x-axis (c(VEGF)) at ng/ml. On the y-axis the change of the BRET ratio dependent on the VEGF concentration is shown in delta milli BRET Units (mBU). Exponential growth is measured in the range of 0.0001 ng/ml up to 0.01 ng/ml, which corresponds to 2 log units of the VEGF concentration.

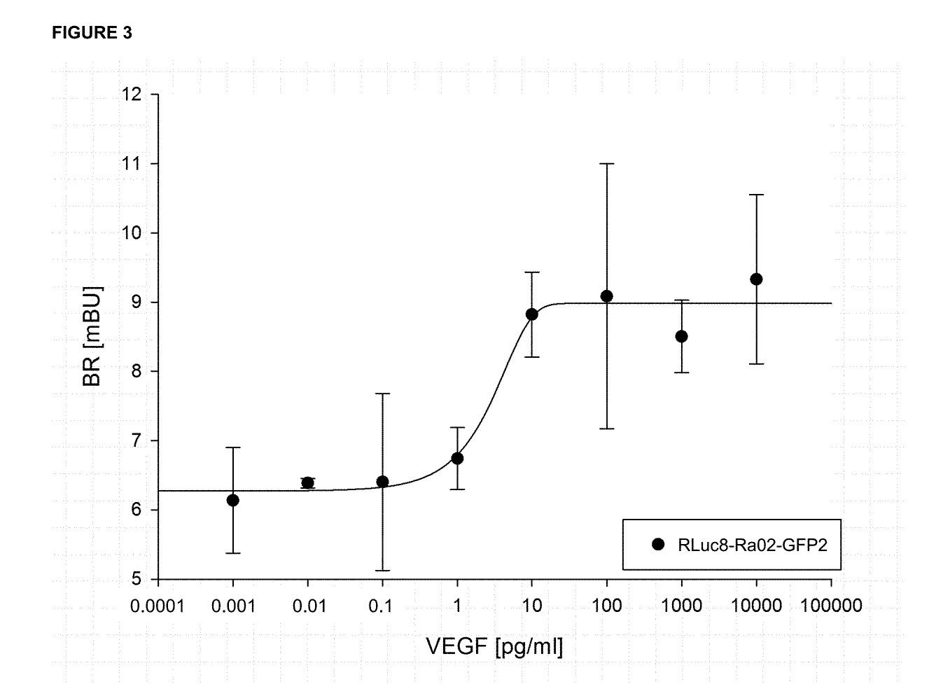

FIG. 4. Change of BRET ratio in dependence of the VEGF concentration. The VEGF concentration is displayed on the x-axis (c(VEGF)) at pg/ml. On the y-axis the change of the BRET ratio (BR) dependent on the VEGF concentration is shown in delta milli BRET Units (mBU). Exponential growth is measured in the range of 0.0001 ng/ml up to 0.01 ng/ml, which corresponds to 2 log units of the VEGF concentration. In one case, the concentration of the biosensor RLuc8-Ra02-GFP2 was 90,000 RLU (relative luciferase units; displayed as black dots), in another case the biosensor concentration was 180,000 RLU (displayed as white triangles).

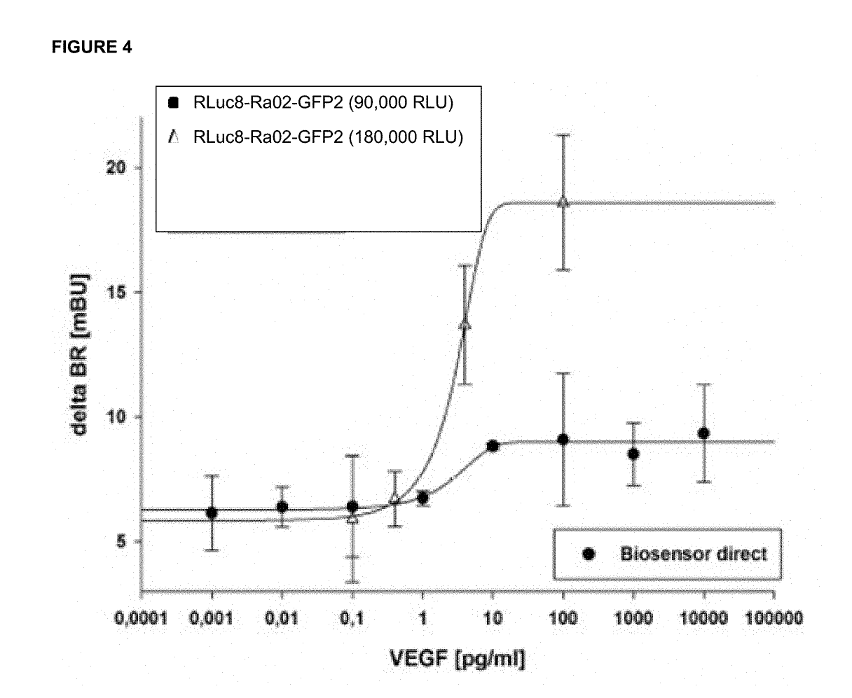

The following sequences referred to herein are shown in the accompanying sequence listing.

### SEQUENCE LISTING

SEQ ID No. 1: RLuc8-Ra02-GFP2 biosensor molecule

SEQ ID No. 2: Vector with TetOn-Ra02 expression cassette

1. A method for measurement and control of intraocular vascular endothelial growth factor (VEGF) concentration, which comprises the steps of:
adding a Renilla luciferase substrate to VEGF-binding biosensor molecules each comprising an anti-VEGF single chain variable fragment (anti-VEGF-scFv) with Renilla luciferase fused to its N-terminus and a fluorescent protein or peptide fused to its C-terminus in a liquid containing VEGF,
measuring bioluminescence resonance energy transfer (BRET) signal depending on binding of prevalent VEGF to the biosensor molecules as an indicator for VEGF concentration, and
inducing expression of anti-VEGF molecules by addition of doxycycline to a vector encoding anti-VEGF molecules that is transduced into eukaryotic cells.

2. The method according to claim 1, wherein the fluorescent protein is GFP2, YFP, eYFP, TurboYFP, or peptides or derivatives or mutants thereof.

3. The method according to claim 1, wherein measurement and control of intraocular VEGF concentration are performed in vivo.

4. The method according to claim 1, wherein the VEGF-binding biosensor and the vector encoding anti-VEGF molecules that is transduced into eukaryotic cells are encapsulated in an eye-implantable, permeable microcapsule, microparticle, microbead, or gel.

5. The method according to claim 4, wherein the eye-implantable, permeable microcapsule, microparticle, microbead, or gel is permeable for VEGF, Renilla luciferase substrate, doxycycline, and anti-VEGF molecules, but is not permeable for VEGF-anti-VEGF antigen-antibody complexes and VEGF bound to VEGF-binding biosensor.

6. The method according to claim 5, wherein the eye-implantable, permeable microcapsule, microparticle, microbead, or gel is made from alginate.

7. The method according to claim 1, wherein measurement of intraocular VEGF concentration is performed in vitro and which comprises the following steps:
addition of a Renilla luciferase substrate to VEGF-binding biosensor molecules each comprising an anti-VEGF single chain variable fragment (anti-VEGF-scFv) with Renilla luciferase fused to its N-terminus and a fluorescent protein or peptide fused to its C-terminus in a liquid containing VEGF,
measurement of bioluminescence resonance energy transfer (BRET) signal depending on binding of VEGF to the biosensor molecules as an indicator for VEGF concentration.

8. The method according to claim 7, wherein the measurement is performed with a sample volume of 1 to 10 μl.

9. The method according to claim 7, wherein the lower detection limit of VEGF concentration is 100 fg/ml and the upper detection limit is 10 ng/ml.

10. A method for diagnosis and/or therapy of VEGF-concentration-related retinal neovascular disorders comprising the steps of:
adding a Renilla luciferase substrate to VEGF-binding biosensor molecules each comprising an anti-VEGF single chain variable fragment (anti-VEGF-scFv) with Renilla luciferase fused to its N-terminus and a fluorescent protein or peptide fused to its C-terminus in a liquid containing VEGF,
measuring bioluminescence resonance energy transfer (BRET) signal depending on binding of prevalent VEGF to the biosensor molecules as an indicator for VEGF concentration, and
inducing expression of anti-VEGF molecules by addition of doxycycline to a vector encoding anti-VEGF molecules that is transduced into eukaryotic cells.

11. A method for diagnosis of VEGF-concentration-related retinal neovascular disorders comprising the steps of:
adding a Renilla luciferase substrate to VEGF-binding biosensor molecules each comprising an anti-VEGF single chain variable fragment (anti-VEGF-scFv) with Renilla luciferase fused to its N-terminus and a fluorescent protein or peptide fused to its C-terminus in a liquid containing VEGF, and
measuring bioluminescence resonance energy transfer (BRET) signal depending on binding of prevalent VEGF to the biosensor molecules as an indicator for VEGF concentration,
wherein measurement of intraocular VEGF concentration is performed in vitro and which comprises the steps of:
adding a Renilla luciferase substrate to VEGF-binding biosensor molecules each comprising an anti-VEGF single chain variable fragment (anti-VEGF-scFv) with Renilla luciferase fused to its N-terminus and a fluorescent protein or peptide fused to its C-terminus in a liquid containing VEGF, and
measuring bioluminescence resonance energy transfer (BRET) signal depending on binding of VEGF to the biosensor molecules as an indicator for VEGF concentration.

12. The method of claim 10 wherein the VEGF concentration-related retinal neovascular disorder is selected from the group consisting of age-related macular degeneration, diabetic macular edema, diabetic retinopathy, retinopathy of prematurity, and retinal vein occlusion.

13. The method of claim 11 wherein the VEGF-concentration-related retinal neovascular disorder is selected from the group consisting of age-related macular degeneration, diabetic macular edema, diabetic retinopathy, retinopathy of prematurity, and retinal vein occlusion.


SEQUENCE LISTING (2 sequences)

SEQ ID NO: 1 - protein, 999 aa, Renilla reniformis
  PEPTIDE (1)..(311): RLuc8 - Renilla luciferase mutant 8
  PEPTIDE (312)..(762): Ra02 - single chain variable fragment (scFv) of ranibizumab
  PEPTIDE (763)..(999): GFP2 - Green fluorescent protein variant
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTHGMDELYR

SEQ ID NO: 2 - DNA, 8736 bp, Artificial Sequence
  Vector
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagctgaagctgatcgatccagcttggtcgagctgatacttcccgtccgccaggggacatgccggcgatgctgaaggtcgcgcgcattcccgatgaagaggccggttaccgcctgttgacctggtgggacgggcagggcgccgcccgagtcttgcctcggcggcgggcgctctgctcatggagcgcgcgtccggggaccttgcacagatagcgtggtccggccagacgacgaggcttgcaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgatgcggccgcgctagtgtcgaccctccatcagagatgtgtcttgtcgcaggatttcggctccactttcttgtccaccttcgtattggagggtttgtggttgacattgcagatgtaggtctgggtccctaagctggaggaaggcacagtaaccacgcttgacagagagtacagaccggatgactggagtacggctggaaacgtatgcacaccagaggttagagcaccactattccaagagacagtcactggttctggaaagtagtccttcaccagacatcccagggctgctgtccctccactggtgctcttagaggagggagccaaggggaatacgcttgggcctttggttgaggcggatgacactgtgaccagggttccttgtccccaaacatcgaaataccagtgacttgtcccgtaatagtacgggtactttgcgcagtagtacacggcagtatcttcggctctcagagagttcatctgcagataggcggttgacttgctagtatcaaggctaaaggtgaacctgcgtttaaagtctgccgcatatgtgggctcgccagtataggtgttaatccagcctacccactctaaccctttgccaggagcttgtcgaacccagttcatcccatagtgtgtgaagtcatagccactagctgcacagctcaaccgtaggcttccgccaggctgaaccagtccaccgccactttcgacgagctgcacctctcctcctccgccgccgccgcattcacctcgattgaaagacttggtcacgggagagcttaagccctgatgagtcacctcacaggcatagaccttgtgtttctcgtaatcggccttactcagggtcagggtgcttgaaaggctgtaggtgctatccttgctgtcctgttctgtgacgctttcctgtgaattgccagattggagtgcattatccactttccattgcaccttcgcttctctgggatagaagttgttgagaaggcaaaccacacttgccgtcccggatttcagctgctcatctgacggtggaaagatgaacacgctaggagcagcaacagtgcgtttgatctctacctttgttccttgtccaaatgtccaaggcacagtgctatactgctgacagtagtaggtggcaaagtcctctggttgtaagcttgagatggtgagagtgaagtctgtgccactgccagatccggaaaacctggagggcacaccactgtgcaagctggaggtgaagtagatcagcactttaggggctttgccaggcttctgctgataccagttcaagtagttggaaatgtcctgggatgcggaacaggtaatcgtgacccgatctccgacggaagcgctcagagaagagggagactgagtcagctgtatgtcatcccctgtactccctggaacccatagaagcagtacccacaacaggagtgtgtccgtctccatgctgcatgcgaattcggatccccgggtaccgagctcgaattcggggccgcggaggctggatcggtcccggtgtcttctatggaggtcaaaacagcgtggatggcgtctccaggcgatctgacggttcactaaacgagctctgcttatataggcctcccaccgtacacgcctactcgacccgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaactagttcgaggtcgacggtatcgataagcttgattcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactataagaatagggccctctaatcgaattcctgcagcccgggggatcgatccttacttagttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggccggcccgccgctttcgcactttagctgtttctccaggccacatatgattagttccaggccgaaaaggaaggcaggttcggctccctgccggtcgaacagctcaattgcttgtctcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggaatgacttggcgttgttccgcagaaagtcttgccatgactcgccttccagggggcagaagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcgccgtttatgactttgctcttgtccagtctagacatggtgaattcaatttaaatcgtaccgagcgactcgacgcgttcgctcgaattaatcaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagttcgagatccccgggtaccgagctcgaattcatcgatgattagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggcc